(19) United States
(12) Patent Application Publication
(10) Pub. No.: US 2015/0122002 A1
(43) Pub. Date: May 7, 2015

(54) APPARATUS FOR MEASURING OF CONTENTS IN EXHAUST GASES

(71) Applicant: SCANIA CV AB, Södertälje (SE)

(72) Inventor: Magnus Mackaldener, Tumba (SE)

(21) Appl. No.: 14/397,765

(22) PCT Filed: Apr. 8, 2013
(86) PCT No.: PCT/SE2013/050376

§ 371 Date: Oct. 29, 2014

(30) Foreign Application Priority Data
May 4, 2012 (SE) 1250447-8

(51) Int. Cl. G01M 15/10; G01N 33/00; F01N 13/00

(52) U.S. Cl. CPC G01M 15/102; F01N 13/008; G01N 33/0037; F01N 2560/026
USPC 73/114.71

(57) ABSTRACT

A device (2) for sampling in the context of measuring the content of the exhaust gases in an exhaust flow (4) in an exhaust line (6) from a combustion engine. A sensor (8) is situated in a measuring chamber (10). At least two gathering tubes (12) situated in the exhaust pipe (6) are provided with apertures (14) which face towards the exhaust flow (4) and are situated in a plane (C-C) which is substantially perpendicular to the direction of the exhaust flow (4). The gathering tubes (12) divert various parts of the exhaust flow to the measuring chamber (10) to make it possible for the sensor (8) to monitor a mean value representing for example the NOx content of the exhaust gases. With a representative mean value for the NOx content of the exhaust gases, an exhaust cleaning system based on SCR technology can be regulated with good accuracy.

### FIELD OF THE INVENTION

The present invention relates to a device according to the preamble of the independent claim. The invention relates more specifically to a device intended primarily to improve the measurement of the NOx content in the exhaust gases from a combustion engine with the object of enabling more accurate regulation of an exhaust post-treatment system.

### BACKGROUND TO THE INVENTION

A combustion engine burns a mixture of air and fuel and generates exhaust gases which inter alia contain nitrogen oxides (NOₓ), carbon dioxide (CO₂), carbon monoxide (CO) and particles. NOₓ is a composite term used to cover primarily nitrogen oxide (NO) and nitrogen dioxide (NO₂). To reduce discharges of harmful constituents, it is usual to provide an exhaust post-treatment system in the exhaust line from the engine. To reduce the NOx content, it is usual in the case of diesel engines for the exhaust post-treatment system to comprise an SCR catalyst in combination with a system for injecting a reducing agent upstream of the catalyst. The reducing agent reacts in the catalyst with the nitrogen oxides and reduces the amounts of NOₓ released into the atmosphere. More specifically, the reducing agent breaks down and forms ammonia (NH₃) which then reacts with NO to form water and nitrogen gas (N₂).

To achieve the NO reduction described, NH₃ has to be stored in the SCR catalyst. For the catalyst to work effectively, the storage level has to be appropriate. In more detail, the NOₓ reduction, or the conversion efficiency, depends on the storage level. To maintain high conversion efficiency in different operating situations, the NH₃ level has to be maintained. However, progressively as the temperature of the catalyst increases, the NH₃ level has to be reduced to avoid NH₃ discharges (i.e. surplus NH₃ being released from the catalyst) which might reduce the catalyst's conversion efficiency.

The exhaust cleaning strategy thus needs to cater for converting sufficient NOₓ while at the same time trying not to inject too much reducing agent, for both environmental and operational economy reasons.

The exhaust gases from an SCR catalyst have different NOₓ concentrations across the catalyst's cross-sectional surface owing to uneven distribution of reducing agent upstream of the catalyst. This results in a skewed NOₓ concentration distribution in the exhaust flow downstream of the catalyst in cases where this is where NOₓ content measurement takes place. Using an NOₓ sensor for feedback and correct control of the amount of reducing agent added with respect to the NOₓ concentration involves the sensor measuring the mean value of the NOₓ content in the exhaust flow, which is difficult where the measurement often takes place at only one point in the exhaust flow.

There are various general techniques for measurement of content in gases.

DD-249096 refers to a device for measurement of various gases, e.g. respiratory air, flowing through a pipe. The three measuring tubes of this device all have the same flow resistance but have gas flowing through them from different points in the pipe to a measuring chamber in which the gases are mixed and the measurement takes place. The gas is then led back to the pipe via an outlet pipe.

DE-1931170U refers to a measuring device with measuring tubes of different lengths which lead gas to a mixing housing provided with a filter which leads the gas to a sensor via a pipe. The gas is then led back via an outlet pipe.

EP-0658756 refers to a measuring device in which an aerosol is led into a measuring chamber via five ducts.

GB 2 135 462 refers to using a pitot tube to lead the exhaust gases in an exhaust flow to a sensor.

U.S. Pat. No. 6,843,104 refers to a solution in which a transverse pipe in the exhaust duct is provided with a number of inlets with the object of taking exhaust gases from different radii and mixing them before they are led to a sensor. The exhaust gases are then led back to the exhaust pipe.

U.S. Pat. No. 7,497,138 refers to a solution largely similar to the system in U.S. Pat. No. 6,843,104.

The object of the present invention is to propose an improved device for sampling of exhaust gases which yields a value for measured content of a substance in the exhaust gases which substantially corresponds to a mean value for the content of the substance in the exhaust gases. This makes it possible for an exhaust cleaning system to be regulated with better accuracy.

### SUMMARY OF THE INVENTION

The above objects are achieved with the invention defined by the independent claim. Preferred embodiments are defined by the dependent claims.

In a device for sampling according to the present invention, exhaust gases are gathered from a plurality of points across the cross-section of the exhaust flow and are mixed before the measurement is performed by a sensor. The result is a more representative mean value of the gas component which is to be measured than by measurement based solely on values from only one measuring point.

The device according to the present invention is particularly suited to being used in the context of measuring the NOₓ content of exhaust gases from combustion engines. It comprises at least two gathering tubes, configured with advantage as pitot tubes, which are provided with apertures facing towards the exhaust flow and are suited to being in close proximity to an exhaust line in order to divert part of the exhaust flow to an NOₓ sensor. Said gathering tubes are all situated in the same plane at right angles to the main direction of the exhaust flow, and said apertures are preferably uniformly distributed in said plane, at a predetermined distance along a radius from the longitudinal axis of the exhaust line.

The device comprises a mixing chamber so arranged as to surround the exhaust line with the flowing exhaust gases. An advantage of such a mixing chamber is that it does not occupy much space and the distance from the various measuring points to the mixing chamber will be minimal in that the gathering tubes may run radially out to the shell surface of the exhaust line. This results in shorter length of lines in the exhaust flow and consequently less flow resistance.

According to the invention, the so-called venturi effect is used to lead the gas back from the measuring chamber to the exhaust line. This effect is achieved by providing the exhaust line with a constriction close to the location where the exhaust gases are led back. The result is better flow and consequently better mixing of the diverted gas.

Further features and advantages are indicated by the attached description exemplifying a number of different embodiments of the invention.

### BRIEF DESCRIPTION OF DRAWINGS

FIG. 1 depicts a schematic cross-sectional view in the longitudinal direction of an exhaust line provided with the device according to a first embodiment of the invention, and

FIGS. 2-5 depict respective schematic cross-sectional views along the line B-B in FIG. 1 of the first, a second, a third and a fourth embodiment of the invention.

### DETAILED DESCRIPTION OF PREFERRED EMBODIMENTS OF THE INVENTION

The same reference numerals are used for the same or similar items in the drawings.

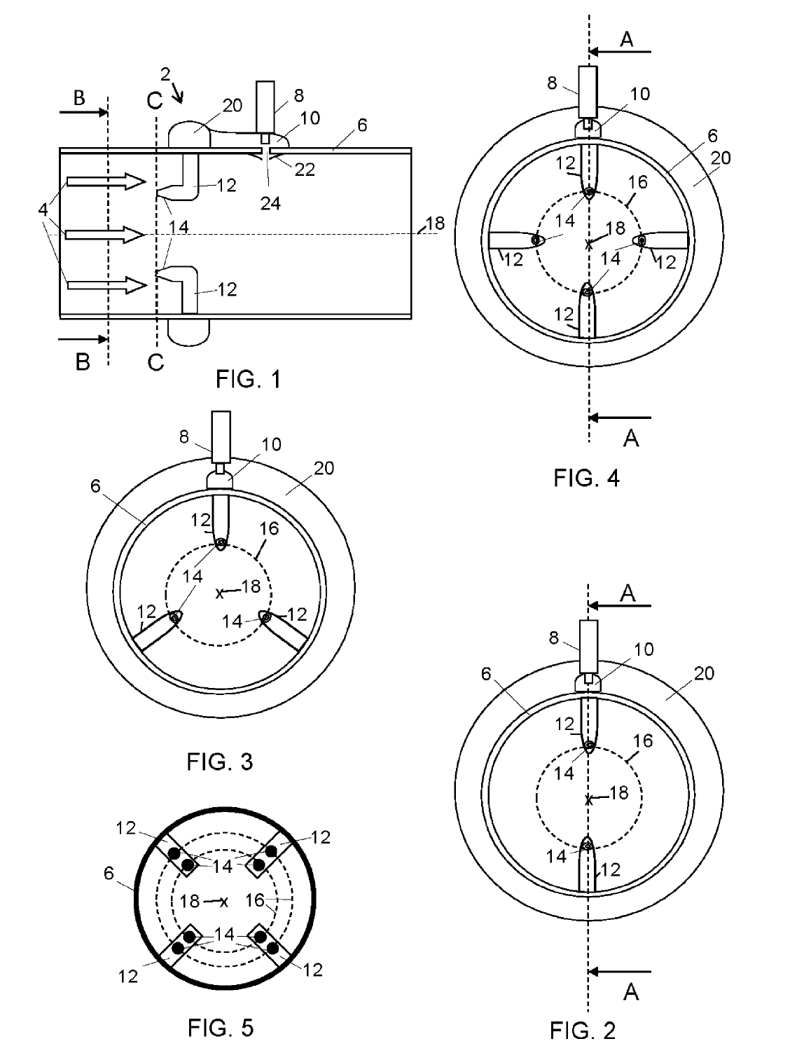

FIGS. 1 and 2 depict a first embodiment of the invention, showing in FIG. 1 a cross-section A-A according to FIG. 2, and in FIG. 2 a cross-section B-B according to FIG. 1.

FIGS. 1 and 2 depict a sampling device 2 suited to being used in the context of measuring the content of exhaust gases in an exhaust flow 4. In one embodiment the sensor is adapted to measuring the NOₓ content of the exhaust gases. Exhaust gases from a combustion engine, e.g. a diesel engine, are led through an exhaust line, exemplified by an exhaust pipe 6, on which the device 2 is located. The device 2 comprises a sensor 8 situated in a measuring chamber 10.

The device 2 further comprises two gathering tubes 12 each partly situated in the exhaust pipe 6 and each comprising at least one aperture 14 facing towards the exhaust flow 4. In FIG. 1, the exhaust flow is represented by arrows. The apertures 14 are preferably all of the same size and situated in a plane C-C which is substantially perpendicular to a main direction of the exhaust flow. The apertures are preferably distributed uniformly in said plane C-C. The gathering tubes 12 are adapted to diverting part of the exhaust flow to said measuring chamber 10 in which the sensor 8 measures the content, e.g. of NOₓ in the exhaust gases diverted to the measuring chamber. The sensor is connected to and forms part of an exhaust cleaning system based on SCR technology (not depicted) whereby measured values from the sensor are used inter alia for dosing of reducing agent.

The fact that the apertures 14 are uniformly distributed in a plane C-C perpendicular to the direction of flow of the exhaust gases means, in an number of different embodiments, that they are evenly distributed on at least one circle 16 at right angles to the direction of the exhaust flow, the centre of which circle coincides with a longitudinal axis 18 of the exhaust pipe 6. See for example the embodiments depicted in FIGS. 2, 3, 4 and 5.

The first embodiment depicted in FIG. 2 relates to a device with two similar gathering tubes 12.

The alternative embodiments depicted in FIGS. 3 and 4 differ only in the number of gathering tubes 12 from the embodiment in FIG. 2. The embodiment depicted in FIG. 3 thus relates to a device 2 with three gathering tubes, and that in FIG. 4 to a device with four gathering tubes. It is essential for there to be at least two gathering tubes, and the greater their number, the better the measuring accuracy, but at the same time the more complex the solution. More than four tubes result in only marginally better measuring accuracy, but if still better accuracy is desired it is instead possible for them to be configured as described below with reference to FIG. 5.

The aperture 14 of each of these gathering tubes is at a predetermined distance from the axis 18 of the exhaust pipe 6. This distance in the drawings is of the order of half of the pipe's radius. The apertures are also uniformly distributed in the circumferential direction.

FIG. 5 depicts an embodiment in which each gathering tube 12 is provided with two apertures 14 along a radius of the respective tube. The embodiment illustrated comprises four gathering tubes. Certain parts, e.g. the measuring chamber with the sensor, are not depicted in FIG. 5. In further embodiments the apertures 14 are likewise uniformly distributed by being at a predetermined distance along a radius from a longitudinal axis 18 of the exhaust pipe 6, and are also uniformly distributed in the circumferential direction.

In the embodiment depicted in FIGS. 1-4, the device 2 comprises a mixing chamber 20 via which the gathering tubes 12 are adapted to leading the diverted exhaust flow 4 to the measuring chamber 10. The mixing chamber is annular in a cross-section of the exhaust pipe 6 and is adapted to surrounding the exhaust pipe. Exhaust gases from the respective apertures 14 are thus led to the mixing chamber 20 which is common to all of the gathering tubes and in which exhaust gases with different contents of NOx will mix. After these exhaust gases with different concentrations of NOx have mixed, they will be monitored by the sensor 8 to arrive at a representative mean value for the NOx content of the exhaust gases in the exhaust line on the basis of different NOx contents in different parts of the exhaust line.

The gathering tubes 12 are all of substantially the same length and have in the exhaust pipe 6 a substantially radial extent relative to the exhaust pipe. This means that the flow from the respective gathering tubes 12 will reach the mixing chamber 20 at approximately the same time, which is advantageous for achieving a correct measured value.

As may be seen in FIG. 1, each of the gathering tubes 12 is inserted partly into the exhaust pipe in the form of a radial portion which by a 90 degree bend changes to an axial direction, the radial outer portion is connected to the mixing chamber 20 and the radial inner axial portion has at its end an aperture 14 which faces towards the exhaust flow.

The exhaust gases diverted via the gathering tubes 12 to the mixing chamber 20 and hence to the measuring chamber 10 have then to be led back to the exhaust pipe 6, which takes place through at least one return pipe 24 adapted to leading exhaust gases back from the measuring chamber to the exhaust pipe 6. In FIG. 1 the return pipe takes the form of a connection from the measuring chamber 10 and is simply an aperture 24 through the wall of the exhaust pipe. The return pipe/aperture 24 is situated in the exhaust pipe at a location downstream of the respective apertures 14 of the gathering tubes.

The exhaust pipe 6 is with advantage provided with a constriction 22 close to the location where the exhaust gases are led back from the measuring chamber 10. The constriction may with advantage take the form of an internal bulge of the exhaust pipe at the aperture 24 through which the exhaust gases are led back. The object of the constriction is to utilise the so-called venturi effect whereby the pressure at the constriction will be less than upstream of the constriction, resulting in a suction effect which contributes to better flow of the exhaust gases past the sensor 8. The constriction need not be at the aperture 24 where the exhaust gases are led back but may for example be anywhere along the inside surface of the exhaust pipe at a cross-section where the aperture 24 is situated.

In applications of the invention for a diesel engine of a heavy vehicle such as a truck, a typical diameter of the exhaust pipe is of the order of 120-130 mm, e.g. 127 mm. The dimensions of the gathering tubes 12 need to be as small as possible in order to minimise the hindrance to the exhaust flow from the engine. Their diameter may for example be within the range 2-10 mm. Their apertures 14 are preferably all of the same size.

The gathering tubes 12 have along substantially the whole of their extent the same inside diameter and their configuration close to the apertures 14 is similar to pitot tubes, which means that their outside diameter in the region round the apertures narrows and thus tapers so that the exhaust gases not led into the apertures can flow past in an advantageous way from a flow perspective.

The gathering tubes 12 may with advantage be situated at a location along the exhaust pipe where the temperature of the exhaust gases is within the range 150-450 degrees C.

Within the scope of the inventive concept it is also possible to omit the mixing chamber 20 and have the exhaust gases led directly from the gathering tubes to the measuring chamber 10. In combination with the constriction 22 described above, this results in good flow past the sensor 8 and consequently good measuring results.

The present invention is not restricted to the preferred embodiments described above. Sundry alternatives, modifications and equivalents may be used.

The invention is described above in an example where the device is part of an SCR system for exhaust cleaning and the measured NOx content is used as a parameter for regulating the injection of a reducing agent. An example of such a reducing agent is a liquid aqueous solution of urea, available commercially as AdBlue®. This liquid is a non-toxic urea solution used to chemically reduce discharges of nitrogen oxide (NOₓ), particularly from diesel-engined heavy vehicles.

In alternative embodiments for alternative purposes the device may be situated in the exhaust line before or after an SCR catalyst, and there may be further components in the exhaust line both before and after the device.

In other applications the device may be used to measure constituents other than NOx in an exhaust flow from a combustion engine. Moreover, the sensor need not be an NOx sensor which directly monitors the concentration of NOx, as it may be any other type of sensor which can indirectly monitor the concentration of NOx. It may for example also be a sensor which monitors the concentration of NO or NO₂. It is also possible for the sensor to be intended to measure the content of hydrocarbons (HC) in the exhaust gases.

The device may in alternative embodiments be used analogously in exhaust lines with cross-sectional shapes other than the circular shape exemplified by an exhaust pipe, e.g. any desired cross-sectional shape.

1. A device for sampling exhaust gases in the context of measuring a content of exhaust gases in an exhaust flow from a combustion engine, wherein the device is situated adjacent to an exhaust line from the engine;
the device comprises a sensor, a measuring chamber in which the sensor is disposed;
at least two apertures into the chamber the apertures facing towards the exhaust flow, in order to divert part of the exhaust flow and lead it to the measuring chamber;
a mixing chamber which is annular and surrounds the exhaust line, the device is configured to leading exhaust gases back from the measuring chamber to the exhaust line at a location downstream of the apertures in the direction of the exhaust flow , the apertures have the form of at least two gathering tubes, each tube is at least partly situated in the exhaust pipe; and
a constriction close to a location in the exhaust pipe where the exhaust gases from the measuring chamber are led back to the exhaust pipe, in order to utilise a venturi effect to improve the exhaust flow past the sensor.

2. The device according to claim 1, in which the apertures are in a plane which is substantially perpendicular to a main direction of the exhaust flow and are uniformly distributed in the plane.

3. The device according to claim 2, in which the apertures are uniformly distributed by being distributed evenly on at least one circle at right angles to the direction of the exhaust flow, and the centre of the circle coincides with a longitudinal axis of the exhaust pipe.

4. The device according to claim 2, in which the apertures are uniformly distributed by being at a predetermined distance along a radius from a central longitudinal axis of the exhaust pipe.

5. The device according to claim 1, further comprising two, three or four similar ones of the gathering tubes.

6. The device according to claim 4, wherein each gathering tube has a radial outer end and from there, each tube extends radially inward towards a radial inner end, and the inner end is angled and extends in a direction towards the direction of the exhaust flow.

7. The device according to claim 1, further comprising at least one return pipe configured for leading exhaust gases back from the measuring chamber to the exhaust line.

8. The device according to claim 1, in which said sensor is configured to measuring NOₓ content of the exhaust gases.
